(19) United States
(12) Patent Application Publication
(10) Pub. No.: US 2008/0167381 A1
(43) Pub. Date: Jul. 10, 2008

(54) 5-Amino-4-Hydroxy-2-Isopropyl-7-[4-Methoxy-3-(3-Methoxypropoxy)Benzyl]-8-Methylnonamides

(76) Inventors: Peter Herold, Basel (CH); Stefan Stutz, Basel (CH); Robert Mah, Muttenz (CH); Vincenzo Tschinke, Binningen (CH)

Correspondence Address: WENDEROTH, LIND & PONACK, L.L.P., 2033 K STREET N. W., SUITE 800, WASHINGTON, DC, 20006-1021, US

(21) Appl. No.: 11/792,676

(22) PCT Filed: Dec. 9, 2005
(86) PCT No.: PCT/EP2005/056623

§ 371 Date: Jun. 8, 2007

(30) Foreign Application Priority Data
Dec. 10, 2004 (CH) 0204904

(51) Int. Cl. A61K 31/165; C07C 233/00; A61P 7/00

(52) U.S. Cl. 514/620; 564/165

(57) ABSTRACT

Compounds of the general formula (I) in which R is heterocyclyl which is bonded via a carbon atom and is mono- or polysubstituted by C₁₋₆-alkyl, trifluoromethyl, nitro, amino, C₁₋₆-alkylamino, di-C₁₋₆-alkylamino, C₂₋₆-alkenyl, C₂₋₆-alkynyl, C₁₋₆-alkoxy, C₁₋₆-alkylcarbonyloxy, C₀₋₆-alkylcarbonylamino, C₁₋₆-alkoxycarbonylamino, hydroxyl, halogen, oxide, cyano, optionally N-mono- or N,N-di-C₁₋₈-alkylated carbamoyl, optionally esterified carboxyl or C₁₋₆-alkylenedioxy, or is substituted by oxo and mono- or polysubstituted by C₁₋₆-alkyl, trifluoromethyl, nitro, amino, C₁₋₆-alkylamino, di-C₁₋₆-alkylamino, C2-6-alkenyl, C₂₋₆-alkynyl, C₁₋₆-alkoxy, C₁₋₆-alkylcarbonyloxy, C₀₋₆-alkylcarbonylamino, C₁₋₆-alkoxycarbonylamino, hydroxyl, halogen, oxide, cyano, optionally N-mono- or N,N-di-C₁₋₈-alkylated carbamoyl, optionally esterified carboxyl or C₁₋₆-alkylenedioxy, and salts, especially pharmaceutically usable salts have renin-inhibiting properties and can be used as medicaments.

The present invention relates to novel alkanamides, to processes for their preparation and to the use of the compounds as medicaments, especially as renin inhibitors.

Alkanamides for use as medicaments are known, for example, from EP 0678503. With regard to renin inhibition in particular, however, there is still a need for highly potent active ingredients. In this context, the improvement of the pharmacokinetic properties is at the forefront. These properties directed towards better bioavailability are, for example, absorption, metabolic stability, solubility or lipophilicity.

The invention therefore provides compounds of the general formula

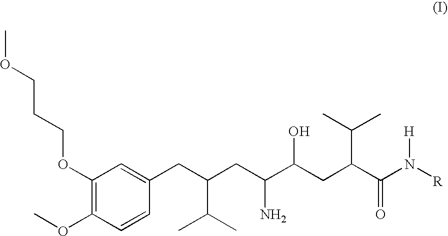

in which
R is heterocyclyl which is bonded via a carbon atom and is mono- or polysubstituted by C₁₋₆-alkyl, trifluoromethyl, nitro, amino, C₁₋₆-alkylamino, di-C₁₋₆-alkylamino, C₂₋₆-alkenyl, C₂₋₆-alkynyl, C₁₋₆-alkoxy, C₁₋₆-alkylcarbonyloxy, C₀₋₆-alkylcarbonylamino, C₁₋₆-alkoxycarbonyl-amino, hydroxyl, halogen, oxide, cyano, optionally N-mono- or N,N-di-C₁₋₈-alkylated carbamoyl, optionally esterified carboxyl or C₁₋₆-alkylenedioxy, or is substituted by oxo and mono- or polysubstituted by C₁₋₆-alkyl, trifluoromethyl, nitro, amino, C₁₋₆-alkylamino, di-C₁₋₆-alkylamino, C₂₋₆-alkenyl, C₂₋₆-alkynyl, C₁₋₆-alkoxy, C₁₋₆-alkylcarbonyloxy, C₀₋₆-alkylcarbonyl-amino, C₁₋₆-alkoxycarbonylamino, hydroxyl, halogen, oxide, cyano, optionally N-mono- or N,N-di-C₁₋₈-alkylated carbamoyl, optionally esterified carboxyl or C₁₋₆-alkylenedioxy, and salts, especially pharmaceutically usable salts thereof.

The term heterocyclyl denotes 4-16-membered, mono- or bicyclic, saturated and unsaturated heterocyclic radicals having 1 to 4 nitrogen and/or 1 or 2 sulphur or oxygen atoms which are substituted. Preference is given to 4-8-membered, particular preference to 5-6-membered, monocyclic radicals which optionally have a 4-8-membered fused-on ring which may be carbocyclic or heterocyclic, and is more preferably a fused-on benzo or pyrido ring. Preferred heterocyclic radicals have, per ring, 1-4 nitrogen atoms, 1-2 oxygen or sulphur atoms, 1-2 nitrogen atoms and 1-2 oxygen atoms, or 1-2 nitrogen atoms and 1-2 sulphur atoms, with at least one carbon atom, preferably 1-7 carbon atoms, being present per ring. Examples of heterocyclyl radicals are pyridyl, thienyl, pyrazinyl, triazolyl, imidazolyl, benzothiazolyl, furyl, pyrimidinyl, quinazolinyl, quinolyl, quinoxalinyl, isoquinolyl, benzo[b]thienyl, isobenzofuranyl, benzimidazolyl, oxazolyl, thiazolyl, indolyl, pyrrolyl, piperidinyl, pyrrolidinyl, pyranyl, tetrahydropyranyl, tetrahydrofuranyl, 1H-pyrrolizinyl, phthalazinyl, [1,5]naphthyridyl, dihydro-2H-benzo[1,4]thiazinyl, dihydro-1H-pyrido[2,3-b][1,4]oxazinyl, 1H-pyrrolo[2,3-b]pyridyl, benzo[1,3]dioxolyl, benzooxazolyl, 3,4-dihydrobenzooxazinyl, 2,3-dihydroindolyl, indazolyl or benzofuranyl.

C₁₋₆-Alkyl may be straight-chain or branched and/or bridged and is, for example, methyl, ethyl, propyl, isopropyl, butyl, isobutyl, sec-butyl, tert-butyl or a pentyl or hexyl group.

C₁₋₆-Alkylamino is, for example, methylamino, ethylamino, propylamino or butylamino.

Di-C₁₋₆-alkylamino is, for example, dimethylamino, N-methyl-N-ethylamino, diethylamino, N-methyl-N-propylamino or N-butyl-N-methylamino.

C₂₋₆-Alkenyl may be straight-chain or branched and is, for example, allyl or vinyl.

C₂₋₆-Alkynyl may be straight-chain or branched and is, for example ethynyl.

C₁₋₆-Alkoxy is, for example, methoxy, ethoxy, propyloxy, isopropyloxy, butyloxy, isobutyloxy, sec-butyloxy, tert-butyloxy, pentyloxy or hexyloxy.

C₁₋₆-Alkoxycarbonylamino is preferably C₂₋₅-alkoxycarbonylamino such as ethoxycarbonyl-amino, propyloxycarbonylamino, isopropyloxycarbonylamino, butyloxycarbonylamino, isobutyloxycarbonylamino, sec-butyloxycarbonylamino or tert-butyloxycarbonylamino.

C₁₋₆-Alkylcarbonyloxy is, for example, acetyloxy, propionyloxy, propylcarbonyloxy, isopropylcarbonyloxy, butylcarbonyloxy, isobutylcarbonyloxy, sec-butylcarbonyloxy, tert-butylcarbonyloxy, pentylcarbonyloxy or hexylcarbonyloxy.

C₀₋₆-Alkylcarbonylamino is, for example, formylamino, acetylamino, propionylamino, propylcarbonylamino, isopropylcarbonylamino, butylcarbonylamino, isobutylcarbonylamino, sec-butylcarbonylamino, tert-butylcarbonylamino, pentylcarbonylamino or hexylcarbonylamino.

Halogen is, for example, fluorine, chlorine, bromine or iodine, preferably fluorine or chlorine.

C₁₋₆-Alkylenedioxy is, for example, methylenedioxy, ethylenedioxy, 1,3-propylenedioxy or 1,2-propylenedioxy.

Optionally N-mono- or N,N-di-C₁₋₈-alkylated carbamoyl is, for example, carbamoyl, methylcarbamoyl, ethylcarbamoyl, N N-dimethylcarbamoyl, N N-diethylcarbamoyl or propylcarbamoyl.

Optionally esterified carboxyl is, for example, carboxyl esterified with C₀₋₆-alkyl, such as carboxyl or C₁₋₆-alkoxycarbonyl.

Depending on the presence of asymmetric carbon atoms, the inventive compounds may be in the form of isomer mixtures, especially as racemates, or in the form of pure isomers, especially of optical antipodes. The invention encompasses all of these forms. Diastereomer mixtures, diastereomeric racemates or mixtures of diastereomeric racemates may be separated by customary methods, for example by column chromatography, thin-layer chromatography, HPLC and the like.

Salts of compounds with salt-forming groups are in particular acid addition salts, salts with bases or, in the presence of a plurality of salt-forming groups, if appropriate also mixed salts or internal salts.

Salts are primarily the pharmaceutically usable or nontoxic salts of compounds of the formula (I). Such salts are formed, for example, from compounds of the formula (I) with an acidic group, for example a carboxyl or sulfo group, and are, for example, the salts thereof with suitable bases, such as nontoxic metal salts derived from metals of group Ia, Ib, IIa and IIb of the Periodic Table of the Elements, for example alkali metal salts, in particular lithium, sodium or potassium salts, alkaline earth metal salts, for example magnesium or calcium salts, and also zinc salts or ammonium salts, and also those salts which are formed with organic amines, such as optionally hydroxy-substituted mono-, di- or trialkylamines, in particular mono-, di- or tri(lower alkyl)amines, or with quaternary ammonium bases, for example methyl-, ethyl, diethyl- or triethylamine, mono-, bis- or tris(2-hydroxy(lower alkyl))amines such as ethanol-, diethanol- or triethanolamine, tris(hydroxymethyl)methylamine or 2-hydroxy-tert-butylamine, N, N-di(lower alkyl)-N-(hydroxy(lower alkyl))amine such as N, N-dimethyl-N-(2-hydroxyethyl)amine or N-methyl-D-glucamine, or quaternary ammonium hydroxides such as tetrabutylammonium hydroxide. The compounds of the formula I having a basic group, for example an amino group, can form acid addition salts, for example with suitable inorganic salts, for example hydrohalic acid such as hydrochloric acid, hydrobromic acid, sulphuric acid with replacement of one or both protons, phosphoric acid with replacement of one or more protons, for example orthophosphoric acid or metaphosphoric acid, or pyrophosphoric acid with replacement of one or more protons, or with organic carboxylic, sulphonic or phosphonic acids, or N-substituted sulphamic acids, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid, isonicotinic acid, and also amino acids, for example the α-amino acids mentioned above, and also methanesulphonic acid, ethanesulphonic acid, 2-hydroxyethanesulphonic acid, ethane-1,2-disulphonic acid, benzenesulphonic acid, 4-toluenesulphonic acid, naphthalene-2-sulphonic acid, 2- or 3-phosphoglycerate, glucose 6-phosphate, N-cyclohexylsulphamic acid (with formation of cyclamates) or with other acidic organic compounds such as ascorbic acid. Compounds of the formula (I) having acidic and basic groups can also form internal salts.

For isolation and purification, pharmaceutically unsuitable salts may also find use.

The compounds of the formula (I) also include those compounds in which one or more atoms are replaced by their stable, non-radioactive isotopes; for example, a hydrogen atom by deuterium.

Prodrug derivatives of the compounds described in the present context are derivatives thereof which, on in vivo application, release the original compound by a chemical or physiological process. A prodrug may be converted to the original compound, for example, when a physiological pH is attained or by enzymatic conversion. Prodrug derivatives may, for example, be esters of freely available carboxylic acids, S- and O-acyl derivatives of thiols, alcohols or phenols, where the acyl group is as defined in the present context. Preference is given to pharmaceutically acceptable ester derivatives which are converted by solvolysis in physiological medium to the original carboxylic acid, for example lower alkyl esters, cycloalkyl esters, lower alkenyl esters, benzyl esters, mono- or disubstituted lower alkyl esters such as lower ω-(amino, mono- or dialkylamino, carboxyl, lower alkoxycarbonyl)alkyl esters or such as lower α-(alkanoyloxy, alkoxycarbonyl or dialkylaminocarbonyl)alkyl esters; as such, pivaloyloxymethyl esters and similar esters are utilized in a conventional manner.

Owing to the close relationship between a free compound, a prodrug derivative and a salt compound, a certain compound in this invention also encompasses its prodrug derivative and salt form, where this is possible and appropriate.

The compound groups specified below are not to be regarded as closed, but rather it is possible in a sensible manner, for example in order to replace general by more specific definitions, to exchange parts of these compound groups with one another or by the definitions given or to omit them.

R is preferably a 5-10-membered mono- or bicyclic radical having at least one nitrogen or oxygen atom and optionally one oxygen or sulphur atom. Most preferably, R is pyridyl, thiazolyl, oxazolyl, indolyl, benzofuranyl, benzothiazolyl or imidazolyl.

R is preferably mono- or disubstituted. Preferred substituents are: C₁₋₆-alkyl, C₁₋₆-alkoxy, C₀₋₆-alkylcarbonylamino, C₁₋₆-alkoxycarbonylamino, trifluoromethyl, nitro, amino, hydroxyl, halogen, oxide, cyano, optionally N-mono- or N,N-di-C₁-C₈-alkylated carbamoyl and optionally esterified carboxyl.

The invention preferably relates to compounds of the formula (I) in which

R is pyridyl, thiazolyl, oxazolyl, indolyl, benzofuranyl, benzothiazolyl or imidazolyl, each of which is bonded via a carbon atom and is mono- or polysubstituted by C₁₋₆-alkyl, C₀₋₆-alkylcarbonylamino, C₁₋₆-alkoxycarbonylamino, C₁₋₆-alkoxy, trifluoromethyl, nitro, amino, hydroxyl, halogen, oxide, cyano, optionally N-mono- or N,N-di-C₁-C₈-alkylated carbamoyl or optionally esterified carboxyl,
and their pharmaceutically usable salts.

Particular preference is given in each case to those compounds of the formula (I) in which at least one asymmetric carbon atom, for example one, two, three or preferably all four asymmetric carbon atoms, of the main chain have the stereochemistry (in each case “S”) shown in the formula (IA), where the substituents are each as defined above, and their pharmaceutically usable salts.

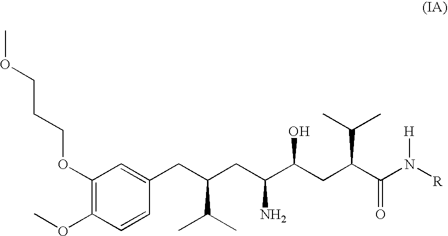

The compounds of the formula (I) or formula (IA) can be prepared in an analogous manner to literature preparation process (see WO 2002/008172 and WO 2002/002508 or literature cited there) (scheme).

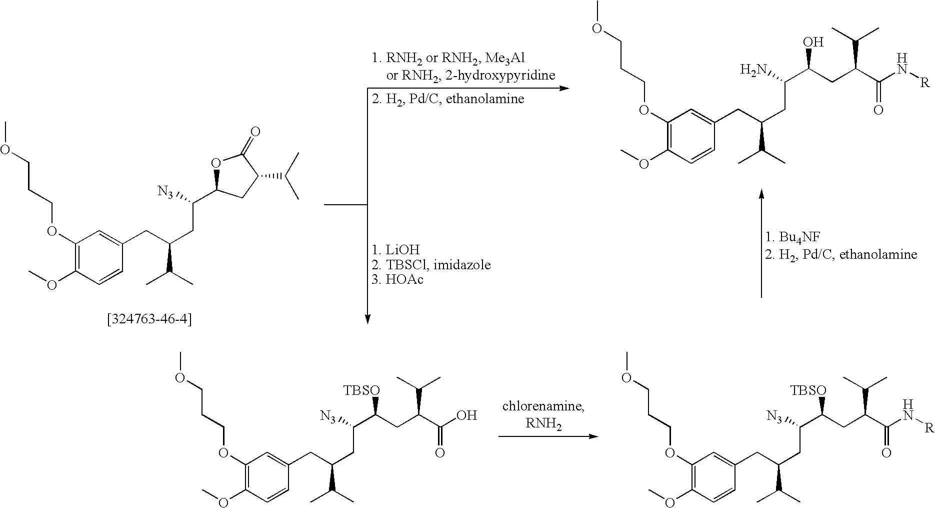

Details of the specific preparation variants can be taken from the examples.

The compounds of the formula (I) may also be prepared in optically pure form. The separation into antipodes may be effected by methods known per se, either preferably at a synthetically early stage by salt formation with an optically active acid, for example (+)- or (−)-mandelic acid, and separation of the diastereomeric salts by fractional crystallization, or preferably at a rather late stage by derivatization with a chiral auxiliary building block, for example (+)- or (−)-camphanoyl chloride, and separation of the diastereomeric products by chromatography and/or crystallization and subsequent cleavage of the bond to the chiral auxiliary. To determine the absolute configuration of the compound present, the pure diastereomeric salts and derivatives may be analysed with common spectroscopic methods, of which X-ray spectroscopy on single crystals constitutes a particularly suitable method.

The compounds of the formula (I) or of the formula (IA) and the pharmaceutically usable salts thereof have inhibiting action on the natural enzyme renin. The latter passes from the kidneys into the blood and therein brings about the cleavage of angiotensinogen to form the decapeptide angiotensin I which is then cleaved in the lung, the kidneys and other organs to the octapeptide angiotensin II. Angiotensin II increases the blood pressure both directly by arterial constriction and indirectly by the release of the hormone aldosterone which inhibits the release of the sodium ion from the adrenal glands, which is associated with a rise in the extracellular liquid volume. This rise can be attributed to the action of angiotensin II itself or of the heptapeptide angiotensin III formed therefrom as a cleavage product. Inhibitors of the enzymatic activity of renin bring about a reduction in the formation of angiotensin I and, as a consequence thereof, the formation of a smaller amount of angiotensin II. The reduced concentration of this active peptide hormone is the immediate cause of the hypotensive action of renin inhibitors.

One experimental method of detecting the action of renin inhibitors is by means of in vitro tests, in which the reduction of the formation of angiotensin I in different systems (human plasma, purified human renin together with synthetic or natural renin substrate) is measured. One in vitro test which is used is the one according to Nussberger et al. (1987) J. Cardiovascular Pharmacol., Vol. 9, p. 39-44 which follows. This test measures the formation of angiotensin I in human plasma. The amount of angiotensin I formed is determined in a subsequent radioimmunoassay. Which action inhibitors have on the formation of angiotensin I is tested in this system by the addition of different concentrations of these substances. The IC₅₀ refers to that concentration of the particular inhibitor which reduces the formation of angiotensin I by 50%. The compounds of the present invention exhibit inhibiting actions in the in vitro systems at minimum concentrations of about 10⁻⁶ to about 10⁻¹⁰ mol/l.

In salt-depleted animals, renin inhibitors bring about a blood pressure decrease. Human renin differs from renin of other species. To test inhibitors of human renin, primates (marmosets, Callithrixjacchus) are used, because human renin and primate renin are substantially homologous in the enzymatically active region. One in vivo test which is used is as follows: the test compounds are tested on normotensive marmosets of both genders and having a body weight of about 350 g which are conscious, able to move freely and in their normal cages. Blood pressure and heart rate are measured using a catheter in the descending aorta and recorded radiometrically. The endogenous release of renin is stimulated by the combination of a 1-week low-salt diet with a single intramuscular injection of furosemide (5-(aminosulphonyl)-4-chloro-2-[(2-furanylmethyl)amino]benzoic acid) (5 mg/kg). 16 hours after the injection of furosemide, the test substances are administered either directly into the femoral artery by means of an injection cannula or into the stomach by gavage as a suspension or solution, and their effect on blood pressure and heart rate is evaluated. The compounds of the present invention effectively reduce blood pressure in the in vivo test described at doses of about 0.003 to about 0.3 mg/kg i.v. and at doses of about 0.3 to about 30 mg/kg p.o.

The compounds of the formula (I), or preferably of the formula (IA), and the pharmaceutically acceptable salts thereof, may find use as medicines, for example in the form of pharmaceutical compositions. The pharmaceutical compositions may be administered enterally, such as orally, for example in the form of tablets, film-coated tablets, sugar-coated tablets, hard and soft gelatin capsules, solutions, emulsions or suspensions, nasally, for example in the form of nasal sprays, rectally, for example in the form of suppositories, or transdermally, for example in the form of ointments or patches. The administration may also be parenteral, such as intramuscular or intravenous, for example in the form of injection solutions.

To prepare tablets, film-coated tablets, sugar-coated tablets and hard gelatin capsules, the compounds of the formula (I) or preferably of the formula (IA) and pharmaceutically acceptable salts thereof may be processed with pharmaceutically inert, inorganic or organic excipients. Such excipients used, for example for tablets, film-coated tablets and hard gelatin capsules, may be lactose, corn starch, or derivatives thereof, talc, stearic acid or salts thereof etc.

Suitable excipients for soft gelatin capsules are, for example, vegetable oils, waxes, fats, semisolid and liquid polyols, etc.

Suitable excipients for preparing solutions and syrups are, for example, water, polyols, sucrose, invert sugar, glucose, etc.

Suitable excipients for injection solutions are, for example, water, alcohols, polyols, glycerol, vegetable oils, bile acids, lecithin, etc.

Suitable excipients for suppositories are, for example, natural or hardened oils, waxes, fats, semisolid or liquid polyols, etc.

The pharmaceutical compositions may additionally also comprise preservatives, solubilizers, viscosity-increasing substances, stabilizers, wetting agents, emulsifiers, sweeteners, colorants, flavourings, salts for altering the osmotic pressure, buffers, coatings or antioxidants. They may also comprise other therapeutically valuable substances.

The present invention further provides for the use of the compounds of the formula (I), or preferably of the formula (IA), and the pharmaceutically acceptable salts thereof, in the treatment or prevention of hypertension, heart failure, glaucoma, myocardial infarction, kidney failure, restenoses or stroke.

The compounds of the formula (I), or preferably of the formula (IA), and the pharmaceutically acceptable salts thereof, may also be administered in combination with one or more agents having cardiovascular action, for example α- and β-blockers such as phentolamine, phenoxybenzamine, prazosin, terazosin, tolazine, atenolol, metoprolol, nadolol, propranolol, timolol, carteolol etc.; vasodilators such as hydralazine, minoxidil, diazoxide, nitroprusside, flosequinan etc.; calcium antagonists such as aminone, bencyclan, diltiazem, fendiline, flunarizine, nicardipine, nimodipine, perhexylene, verapamil, gallopamil, nifedipine etc.; ACE inhibitors such as cilazapril, captopril, enalapril, lisinopril etc.; potassium activators such as pinacidil; anti-serotoninergics such as ketanserin; thromboxane-synthetase inhibitors; neutral endopeptidase inhibitors (NEP inhibitors); angiotensin II antagonists; and also diuretics such as hydrochlorothiazide, chlorothiazide, acetazolamide, amiloride, bumetanide, benzthiazide, ethacrynic acid, furosemide, indacrinone, metolazone, spironolactone, triamteren, chlorthalidone etc.; sympatholytics such as methyldopa, clonidine, guanabenz, reserpine; and other agents which are suitable for the treatment of hypertension, heart failure or vascular diseases in humans and animals which are associated with diabetes or renal disorders such as acute or chronic renal failure. Such combinations may be employed separately or in preparations which comprise a plurality of components.

Further substances which can be used in combination with the compounds of the formula (I) or (IA) are the compounds of classes (i) to (ix) on page 1 of WO 02/40007 (and also the preferences and examples further listed therein) and the substances specified on pages 20 and 21 of WO 03/027091.

The dose may vary within wide limits and has of course to be adapted to the individual circumstances in each individual case. In general, for oral administration, a daily dose of about 3 mg to about 3 g, preferably about 10 mg to about 1 g, for example about 300 mg, per adult (70 kg), divided into preferably 1-3 individual doses which may, for example, be of equal size, may be appropriate, although the upper limit specified may also be exceeded if this should be found to be appropriate; typically, children receive a lower dose according to their age and body weight.

The examples which follow illustrate the present invention. All temperatures are reported in degrees Celsius, pressures in mbar. Unless stated otherwise, the reactions take place at room temperature. The abbreviation “Rf=xx (A)” means, for example, that the Rf value xx is determined in the solvent system A. The ratio of solvents relative to one another is always reported in parts by volume. Chemical names for end products and intermediates were generated with the aid of the program AutoNom 2000 (automatic nomenclature).

HPLC gradients on Hypersil BDS C-18 (5 μm); column: 4×125 mm

I 90% water*/10% acetonitrile*/to 0% water*/100% acetonitrile*/in 5 minutes+2.5 minutes (1.5 ml/min)
II 95% water*/5% acetonitrile*/to 0% water*/100% acetonitrile*/in 40 minutes (0.8 ml/min)
*contains 0.1% trifluoroacetic acid

The following abbreviations are used:

Rf ratio of distance traveled by a substance to separation of the eluent front from the start point in thin-layer chromatography
Rt retention time of a substance in HPLC (in minutes)
m.p. melting point (temperature)

### General Method A: (Azide Reduction)

A solution of 1 mmol of “azide derivative” in 10-20 ml of ethanol and ethanolamine (1 equiv.) is hydrogenated in the presence of 200-400 mg of 10% Pd/C (moist) at 0° C. over 1-3 hours. The reaction mixture is clarified by filtration and the catalyst is washed with ethanol. The filtrate is concentrated by evaporation. The title compound is obtained from the residue by means of flash chromatography (SiO₂ 60 F).

### General Method B: (Lactone Amidation I)

A mixture of 1 mmol of “lactone”, “amine” (10-30 equiv.) and 2-hydroxypyridine (1 equiv.) is stirred at 65° C. over 2-24 hours. The reaction mixture is cooled to room temperature, concentrated by evaporation, admixed with 1M aqueous sodium hydrogencarbonate solution and extracted with tert-butyl methyl ether (2×). The combined organic phases are washed with water and brine, dried over sodium sulphate and concentrated. The title compound is obtained from the residue by means of flash chromatography (SiO₂ 60 F).

### General Method C: (Lactone Amidation II)

A solution of 1.1 mmol of trimethylaluminium solution (2 M in heptane) at −78° C. is admixed with a solution of 1.2 mmol of “amine” in 1-2 ml of toluene. The reaction mixture is warmed to room temperature, stirred for a further 30-60 minutes and subsequently concentrated by evaporation. The residue is admixed with a solution of 1 mmol of “lactone” in 2 ml of toluene and stirred at 80° C. for 2-4 hours. The reaction mixture is cooled to room temperature, admixed with 10 ml of 1N HCl and then stirred for a further 30 minutes. The reaction mixture is diluted with brine and extracted with toluene (2×)—the combined organic phases are dried over sodium sulphate and concentrated by evaporation. The title compound is obtained from the residue by means of flash chromatography (SiO₂ 60 F).

### General Method D: (Desilylation)

A solution of 1 mmol of “silyl ether” in 10-15 ml of tetrahydrofuran at 0° C. is admixed with 1.5 mmol of tetrabutylammonium fluoride solution (1M in tetrahydrofuran). The reaction mixture is stirred at room temperature over 2-4 hours, poured onto 1M aqueous sodium hydrogencarbonate solution and extracted with tert-butyl methyl ether (2×). The combined organic phases are washed with brine, dried over sodium sulphate and concentrated by evaporation. The title compound is obtained from the residue by means of flash chromatography (SiO₂ 60 F).

### General Method E: (Chlorenamine Coupling)

A solution of 1 mmol of “acid” in 10 ml of dichloromethane at 0° C. is admixed with 1.2-1.8 mmol of (1-chloro-2-methylpropenyl)dimethylamine(chlorenamine). After 2-4 hours, the reaction mixture is concentrated by evaporation and the residue is dissolved in 6 ml of dichloromethane—this solution is added dropwise to the solution of 1.25 mmol of “amine” and 1.1 mmol of triethylamine in 6 ml of dichloromethane at 0° C. over 2-10 minutes. The reaction mixture is stirred at room temperature over 1-2 hours, poured onto water and extracted with tert-butyl methyl ether (2×). The combined organic phases are washed with brine, dried over sodium sulphate and concentrated by evaporation. The title compound is obtained from the residue by means of flash chromatography (SiO₂ 60 F).

### General Method F: (Lactone Opening/Silylation)

A solution of 1 mmol of “lactone” in 5 ml of dioxane is admixed with 5 ml of water and 1.1 mmol of lithium hydroxide monohydrate. After 4-6 hours, the reaction mixture is admixed with ice and 1M aqueous citric acid solution and extracted with tert-butyl methyl ether (3×). The combined organic phases are washed with cold water and cold brine, dried over sodium sulphate and concentrated by evaporation at room temperature. The residue is immediately dissolved in 8 ml of N,N-dimethylformamide and then admixed with 5 mmol of tert-butyl-chlorodimethylsilane and 8.8 mmol of imidazole. After 10-20 hours, the reaction mixture is concentrated by evaporation—the residue is admixed with diethyl ether and water, adjusted to pH 4 with 1M aqueous citric acid solution, and then the organic phase is removed. The aqueous phase is extracted once more with diethyl ether (3×)—the combined organic phases are washed with brine, dried over sodium sulphate and concentrated by evaporation. The residue is dissolved in 3 ml of tetrahydrofuran and admixed successively with 3 ml of water and 9 ml of acetic acid. After 3-4 hours, the reaction mixture is poured onto ice-water and extracted with diethyl ether (2×)—the combined organic phases are washed with water and brine, dried over sodium sulphate and concentrated by evaporation. The title compound is obtained from the residue by means of flash chromatography (SiO₂ 60 F).

### General Method G: (N-Cbz Protection)

A solution of 1 mmol of “amine” in 10 ml of ethyl acetate is admixed with 10 ml of saturated sodium carbonate solution and admixed at 0° C. with 1.1 mmol of benzyl chloroformate. After 1-3 hours, the phases of the reaction mixture are separated. The aqueous phase is extracted with ethyl acetate (2×)—the combined organic phases are washed with 1M sodium hydrogen-carbonate solution and brine, dried over sodium sulphate and concentrated by evaporation. The title compound is obtained from the residue by means of flash chromatography (SiO₂ 60 F).

### General Method H: (Pyridine Oxidation)

A solution of 1 mmol of “pyridine” in 30 ml of dichloromethane is admixed with 2 mmol of m-chloroperbenzoic acid and stirred at room temperature for 2 hours. The mixture is poured onto 1M NaOH and extracted with tert-butyl methyl ether (3×). The combined organic phases are washed with 1M NaOH, water and brine, dried over sodium sulphate and concentrated by evaporation. The title compound is obtained from the residue by means of flash chromatography (SiO₂ 60 F).

### General Method I: (N-Cbz Deprotection)

A solution of 1 mmol of “N-Cbz derivative” in 10-20 ml of ethanol and ethanolamine (1 equiv.) is hydrogenated in the presence of 200-400 mg of 10% Pd/C (moist) at 0° C. over 1-3 hours. The reaction mixture is clarified by filtration and the catalyst is washed with ethanol. The filtrate is concentrated by evaporation. The title compound is obtained from the residue by means of flash chromatography (SiO₂ 60 F).

### EXAMPLE 1

### N-(5-Chloropyridin-2-yl)-5(S)-amino-4(S)-hydroxy-2(S)-isopropyl-7(S)-[4-methoxy-3-(3-methoxypropoxy)benzyl]-8-methylnonamide

Analogously to method A, 0.088 g of N-(5-chloropyridin-2-yl)-5(S)-azido-4(S)-hydroxy-2(S)-isopropyl-7(S)-[4-methoxy-3-(3-methoxypropoxy)benzyl]-8-methylnonanamide is used to obtain the title compound as a colourless oil. Rf=0.30 (200:20:1 dichloromethane-methanol-25% conc. ammonia); Rt=4.51 (gradient 1).

The starting material is prepared as follows:

### a) N-(5-Chloropyridin-2-yl)-5(S)-azido-4(S)-hydroxy-2(S)-isopropyl-7(S)-[4-methoxy-3-(3-methoxypropoxy)benzyl]-8-methylnonanamide

Analogously to method D, 0.11 g of N-(5-chloropyridin-2-yl)-5(S)-azido-4(S)-(tert-butyldimethylsilanyloxy)-2(S)-isopropyl-7(S)-[4-methoxy-3-(3-methoxypropoxy)benzyl]-8-methylnonanamide is reacted. The title compound is obtained as a colourless oil. Rf=0.13 (1:2 EtOAc-heptane); Rt=5.61 (gradient 1).

### b) N-(5-Chloropyridin-2-yl)-5(S)-azido-4(S)-(tert-butyldimethylsilanyloxy)-2(S)-isopropyl-7(S)-[4-methoxy-3-(3-methoxypropoxy)benzyl]-8-methylnonanamide

Analogouosly to method E, 0.220 g of 5(S)-azido-4(S)-(tert-butyldimethylsilanyloxy)-2(S)-isopropyl-7(S)-[4-methoxy-3-(3-methoxypropoxy)benzyl]-8-methylnonanoic acid and 0.060 g of 5-chloropyridin-2-ylamine are reacted. The title compound is obtained as a colourless oil. Rf=0.40 (1:2 EtOAc-heptane).

### c) 5(S)-Azido-4(S)-(tert-butyldimethylsilanyloxy)-2(S)-isopropyl-7(S)-[4-methoxy-3-(3-methoxypropoxy)benzyl]-8-methylnonanoic acid

Analogously to method F. 0.933 g of 5(S)-{1(S)-azido-3(S)-[4-methoxy-3-(3-methoxypropoxy)benzyl]-4-methylpentyl}-3(S)-isopropyldihydrofuran-2-one [324763-46-4] is reacted.

The title compound is obtained as a yellowish oil. Rf=0.40 (1:1 EtOAc-heptane); Rt=6.54 (gradient 1).

### EXAMPLE 2

### N-(1-Oxypyridin-4-yl)-5(S)-amino-4(S)-hydroxy-2(S)-isopropyl-7(S)-[4-methoxy-3-(3-methoxypropoxy)benzyl]-8-methylnonanamide

Analogously to method I, 0.150 g of benzyl [(1S,2S,4S)-2-hydroxy-1-{(S)-2-[4-methoxy-3-(3-methoxypropoxy)benzyl]-3-methylbutyl}-5-methyl-4-(1-oxypyridin-4-ylcarbamoyl)hexyl]-carbamate is used to obtain the title compound as a yellow foam. Rf=0.53 (40:10:1 dichloromethane-methanol-25% conc. ammonia); Rt=3.33 (gradient 1).

The starting material is prepared as follows:

### a) Benzyl [(1S,2S,4S)-2-hydroxy-1-[(S)-2-[4-methoxy-3-(3-methoxypropoxy)benzyl]-3-methyl butyl]-5-methyl-4-(1-oxypyridin-4-ylcarbamoyl)hexyl]carbamate

Analogously to method H. 0.270 g of benzyl [(1S,2S,4S)-2-hydroxy-1-{(S)-2-[4-methoxy-3-(3-methoxypropoxy)benzyl]-3-methylbutyl}-5-methyl-4-(pyridin-4-ylcarbamoyl)hexyl]carbamate is used to obtain the title compound as a white foam. Rf=0.13 (200:10:1 dichloromethane-methanol-25% conc. ammonia); Rt=4.48 (gradient 1).

### b) Benzyl [(1S,2S,4S)-2-hydroxy-1-[(S)-2-[4-methoxy-3-(3-methoxypropoxy)benzyl]-3-methyl butyl]-5-methyl-4-(pyridin-4-ylcarbamoyl)hexyl]carbamate

Analogously to method G. 0.231 g of N-(pyridine-4-yl)-(2S,4S,5S,7S)-5-amino-4-hydroxy-2-isopropyl-7-[4-methoxy-3-(3-methoxypropoxy)benzyl]-8-methylnonanamide is used to obtain the title compound as a slightly yellowish foam. Rf=0.45 (200:10:1 dichloromethane-methanol-25% conc. ammonia); Rt=4.55 (gradient 1).

### c) N-(Pyridine-4-yl)-(2S,4S,5S,7S)-5-amino-4-hydroxy-2-isopropyl-7-[4-methoxy-3-(3-methoxypropoxy)benzyl]-8-methylnonanamide

Analogously to method A, 0.231 g of N-(pyridine-4-yl)-(2S,4S,5S,7S)-5-azido-4-hydroxy-2-isopropyl-7-[4-methoxy-3-(3-methoxypropoxy)benzyl]-8-methylnonanamide is used to obtain the title compound as a slightly yellowish foam. Rf=0.16 (200:10:1 dichloromethane-methanol-25% conc. ammonia); Rt=3.34 (gradient 1).

### d) N-(Pyridine-4-yl)-(2S,4S,5S,7S)-5-azido-4-hydroxy-2-isopropyl-7-[4-methoxy-3-(3-methoxypropoxy)benzyl]-8-methylnonanamide

Analogously to method C, 0.4665 g of 5(S)-{1(S)-azido-3(S)-[4-methoxy-3-(3-methoxypropoxy)benzyl]-4-methylpentyl}-3(S)-isopropyldihydrofuran-2-one [324763-46-4] is used to obtain the title compound as a white solid. Rf=0.13 (95:5 dichloromethane-methanol); Rt=4.54 (gradient 1).

The processes described in examples 1 and 2 are used analogously to prepare the following compounds:

### EXAMPLES

3 N-(4-Methylpyridin-2-yl)-5(S)-amino-4(S)-hydroxy-2(S)-isopropyl-7(S)-[4-methoxy-3-(3-methoxypropoxy)benzyl]-8-methylnonanamide
4 N-(5-Methylpyridin-2-yl)-5(S)-amino-4(S)-hydroxy-2(S)-isopropyl-7(S)-[4-methoxy-3-(3-methoxypropoxy)benzyl]-8-methylnonanamide
5 N-(3-Methylpyridin-2-yl)-5(S)-amino-4(S)-hydroxy-2(S)-isopropyl-7(S)-[4-methoxy-3-(3-methoxypropoxy)benzyl]-8-methylnonanamide
6 N-(6-Methylpyridin-3-yl)-5(S)-amino-4(S)-hydroxy-2(S)-isopropyl-7(S)-[4-methoxy-3-(3-methoxypropoxy)benzyl]-8-methylnonanamide
7 N-(2-Methylpyridin-4-yl)-5(S)-amino-4(S)-hydroxy-2(S)-isopropyl-7(S)-[4-methoxy-3-(3-methoxypropoxy)benzyl]-8-methylnonanamide
8 N-(6-Methylpyridin-2-yl)-5(S)-amino-4(S)-hydroxy-2(S)-isopropyl-7(S)-[4-methoxy-3-(3-methoxypropoxy)benzyl]-8-methylnonanamide
9 N-(5-Fluoropyridin-2-yl)-5(S)-amino-4(S)-hydroxy-2(S)-isopropyl-7(S)-[4-methoxy-3-(3-methoxypropoxy)benzyl]-8-methylnonanamide
10 N-(3,5-Difluoropyridin-2-yl)-5(S)-amino-4(S)-hydroxy-2(S)-isopropyl-7(S)-[4-methoxy-3-(3-methoxypropoxy)benzyl]-8-methylnonanamide
11 N-(5-Cyanopyridin-2-yl)-5(S)-amino-4(S)-hydroxy-2(S)-isopropyl-7(S)-[4-methoxy-3-(3-methoxypropoxy)benzyl]-8-methylnonanamide
12 N-(3-Fluoropyridin-2-yl)-5(S)-amino-4(S)-hydroxy-2(S)-isopropyl-7(S)-[4-methoxy-3-(3-methoxypropoxy)benzyl]-8-methylnonanamide
13 N-(6-Fluoropyridin-2-yl)-5(S)-amino-4(S)-hydroxy-2(S)-isopropyl-7(S)-[4-methoxy-3-(3-methoxypropoxy)benzyl]-8-methylnonanamide
14 N-(6-Trifluoromethylpyridin-3-yl)-5(S)-amino-4(S)-hydroxy-2(S)-isopropyl-7(S)-[4-methoxy-3-(3-methoxypropoxy)benzyl]-8-methylnonanamide
15 N-(2-Fluoropyridin-3-yl)-5(S)-amino-4(S)-hydroxy-2(S)-isopropyl-7(S)-[4-methoxy-3-(3-methoxypropoxy)benzyl]-8-methylnonanamide
16 N-(3-Fluoropyridin-4-yl)-5(S)-amino-4(S)-hydroxy-2(S)-isopropyl-7(S)-[4-methoxy-3-(3-methoxypropoxy)benzyl]-8-methylnonanamide
17 N-(2-Fluoropyridin-4-yl)-5(S)-amino-4(S)-hydroxy-2(S)-isopropyl-7(S)-[4-methoxy-3-(3-methoxypropoxy)benzyl]-8-methylnonanamide
18 N-(6-Fluoropyridin-3-yl)-5(S)-amino-4(S)-hydroxy-2(S)-isopropyl-7(S)-[4-methoxy-3-(3-methoxypropoxy)benzyl]-8-methylnonanamide
19 N-(1-Oxypyridin-2-yl)-5(S)-amino-4(S)-hydroxy-2(S)-isopropyl-7(S)-[4-methoxy-3-(3-methoxypropoxy)benzyl]-8-methylnonanamide
20 N-(1-Oxypyridin-3-yl)-5(S)-amino-4(S)-hydroxy-2(S)-isopropyl-7(S)-[4-methoxy-3-(3-methoxypropoxy)benzyl]-8-methylnonanamide

1. Compound of the formula 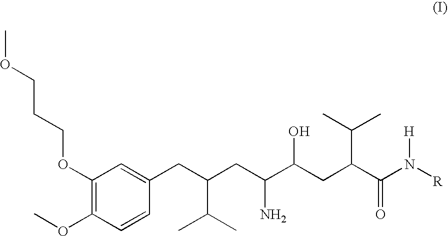
in which
R is heterocyclyl which is bonded via a carbon atom and is mono- or polysubstituted by C₁₋₆-alkyl, trifluoromethyl, nitro, amino, C₁₋₆-alkylamino, di-C₁₋₆-alkylamino, C₂₋₆-alkenyl, C₂₋₆-alkynyl, C₁₋₆-alkoxy, C₁₋₆-alkylcarbonyloxy, C₀₋₆-alkylcarbonylamino, C₁₋₆-alkoxycarbonylamino, hydroxyl, halogen, oxide, cyano, optionally N-mono- or N,N-di-C₁₋₈-alkylated carbamoyl, optionally esterified carboxyl or C₁₋₆-alkylenedioxy, or is substituted by oxo and mono- or polysubstituted by C₁₋₆-alkyl, trifluoromethyl, nitro, amino, C₁₋₆-alkylamino, di-C₁₋₆-alkylamino, C₂₋₆-alkenyl, C₂₋₆-alkynyl, C₁₋₆-alkoxy, C₁₋₆-alkylcarbonyloxy, C₀₋₆-alkylcarbonylamino, C₁₋₆-alkoxycarbonylamino, hydroxyl, halogen, oxide, cyano, optionally N-mono- or N,N-di-C₁₋₈-alkylated carbamoyl, optionally esterified carboxyl or C₁₋₆-alkylenedioxy,
and its salt, especially pharmaceutically usable salt.

2. Compound according to claim 1 of the formula 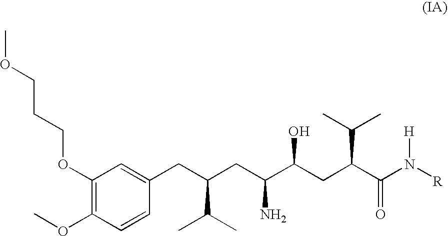
in which R is as defined in claim 1.

3. Compound according to claim 1, in which
R is a 5-10-membered mono- or bicyclic radical having at least one nitrogen or oxygen atom and optionally one oxygen or sulphur atom.

4. Compound according to claim 1, in which
R is mono- or disubstituted by C₁₋₆-alkyl, C₀₋₆-alkylcarbonylamino, C₁₋₆-alkoxycarbonylamino, C₁₋₆-alkoxy, trifluoromethyl, nitro, amino, hydroxyl, halogen, oxide, cyano, optionally N-mono- or N,N-di-C₁₋₈-alkylated carbamoyl or optionally esterified carboxyl.

5. Compound according to claim 1, in which
R is pyridyl, thiazolyl, oxazolyl, indolyl, benzofuranyl, benzothiazolyl or imidazolyl, each of which is bonded via a carbon atom and is substituted by C₁₋₆-alkyl, C₀₋₆-alkylcarbonylamino, C₁₋₆-alkoxycarbonylamino, C₁₋₆-alkoxy, trifluoromethyl, nitro, amino, hydroxyl, halogen, oxide, cyano, carbamoyl or carboxyl.

6. Use of a compound of the general formula (I) according to claim 1 for producing a medicament.

7. Use of a compound of the general formula (I) according to claim 1 for producing a human medicament for the prevention, for the retardation of progression or for the treatment of hypertension, heart failure, glaucoma, myocardial infarction, kidney failure, restenoses or stroke.

8. Method for the prevention, for the retardation of progression or for the treatment of hypertension, heart failure, glaucoma, myocardial infarction, kidney failure, restenoses or stroke, in which a therapeutically effective amount of a compound of the general formula (I) according to claim 1 is used.

9. Pharmaceutical preparation comprising a compound of the general formula (I) according to claim 1, and also customary excipients.

10. Pharmaceutical combination in the form of a preparation or of a kit composed of individual components, consisting
a) of a compound of the general formula (I) according to claim 1 and
b) at least one drug form whose active ingredient has cardiovascular action.
